(19) United States
(12) Patent Application Publication
(10) Pub. No.: US 2010/0269582 A1
(43) Pub. Date: Oct. 28, 2010

(54) Method and a System for Lameness Recognition

(76) Inventors: Tal Bareket, Kiryat Tivon (IL); Noam Pinski, Kineret-Moshava (IL)

Correspondence Address: MARTIN D. MOYNIHAN d/b/a PRTSI, INC., P.O. BOX 16446, ARLINGTON, VA, 22215, US

(21) Appl. No.: 12/225,347

(22) PCT Filed: Mar. 19, 2007
(86) PCT No.: PCT/IL07/00352

§ 371 Date: Nov. 5, 2008

(30) Foreign Application Priority Data
Mar. 21, 2006 (IL) 174449

(51) Int. Cl. A61B 5/11

(52) U.S. Cl. 73/172

(57) ABSTRACT

A method and a system for recognizing a lameness of an animal are disclosed.

The lameness recognition is done by detecting position changes of a defined element on the animal's body, during a sequence of at least two said animal's steps; comparing the position changes of an animal's step with the position changes of the subsequent step of the animal; or/and comparing the position changes with a recorded position changes information of same element of same animal.

### FIELD OF THE INVENTION

The present invention relates to the field of dairy management and animals' healthy. More specifically, the present invention relates to an animal's lameness diagnosis in a dairy.

### BACKGROUND OF THE INVENTION

Lameness is a spread disease in a dairy. In a day-by-day work in a dairy that contains large number of ruminants, it is very difficult to recognize lameness animal until it became very severe. On the other hand, medical treatment in the early start of the lameness is very efficient.

Therefore, an objective method and automatic system for lameness recognition that can be applied on a large number of animals is essential.

The present invention is based on the behavior of a lameness animal in contrast with a healthy one. Lameness caused by a disease or a wound in the leg of the animal, which causes pain while the animal, is step on the diseased or wounded leg. Therefore the lameness animal, while walking, instinctively shortens the stepping time on the diseased or wounded leg by accelerating the movement of the other leg. The main object of the present invention is the recognition of the walking behavior changes of a lameness animal.

The present invention is a novel method and system for recognizing animal's lameness.

### SUMMARY OF THE INVENTION

The present invention is a method and a system for animal's lameness recognition.

According to the teachings of the present invention there is provided a method for recognizing a lameness of an animal, the method is comprised of the following steps:
detecting position changes of a defined element on the animal's body, during a sequence of at least two the animal's steps;
comparing the position changes of an animal's step with the position changes of the subsequent step of the animal; or/and
comparing the position changes with a recorded position changes information of same element of same animal.

According to a preferred embodiment the method is provided, wherein lameness is defined as a significant change between the element's changes position during the animal's step and the element's change of the subsequent step and/or significant changes between the element's changes position during the steps and the recorded element's changes.

According to another preferred embodiment the provided method further includes the step of comparing between the distance of the animal's step and the distance of the consequent step.

According to another preferred embodiment the provided method further includes the step of comparing between the back's arching of the animal with a recorded back's arching of the same animal.

According to another preferred embodiment the method is provided, wherein the element is the hip of the animal.

According to another preferred embodiment the method is provided, wherein the element is the withers of the animal.

According to another preferred embodiment the method is provided, wherein the element changes is at least one of the followings:
the angle between each two legs of the animal;
the hoofs distance of each two legs of the animal;
the inclination of each animal's leg; and/or
the leg's folding angle of each animal's leg.

According to another preferred embodiment the method is provided, wherein the detection is done by a plurality of sequential sampling of the element' position during each the animal's step to obtain a repeating wave for each two steps of the animal and wherein lameness is defined as a significant pattern's change between the two halves of the repeating wave.

According to another preferred embodiment the method is provided, wherein the position changes are a plurality of height or lowness measurement according to a reference.

According to another preferred embodiment the method is provided, wherein the detection is done by a sensor or by measuring device.

According to another preferred embodiment the method is provided, wherein a processor does the comparison.

According to another preferred embodiment the method is provided, wherein the position changes are drawn by a drawing means or displayed, and the comparison in done visually.

According to another aspect of the present invention it is provided an animal' lameness recognizing system that is comprised of:
an animal's ID means;
a detecting means for tracking position changes of an animals' body element during at lest two steps of the animal;
a memory for storing the tracking information and other information; and
a controller operative for:
   receiving the ID of an animal from the ID means;
   receiving the element's tracking information from the tracking means or from the memory and obtaining an element's position changes repeating-wave of two following steps;
   analyzing the repeating-wave to recognize lameness; and
   providing announcement and animal ID, when lameness is recognized.

According to a preferred embodiment the system, according to the present invention, is provided wherein the repeating-wave is analyzed by the way of:
applying mathematical technique to divide the repeating-wave into two cycle halves;
comparing the pattern of the two cycle halves; and
defining lameness in the case that the two cycle halves are significantly differ.

According to another preferred embodiment the system is provided, wherein the repeating-wave is analyzed by the way of:
comparing the pattern of the repeating-wave with a pre recorded repeating-wave of same animal's body element of same animal in a non-lameness condition.

According to another preferred embodiment the system is provided, wherein the element changes is at least one of the followings:
the angle between each two legs of the animal;
the hoofs distance of each two legs of the animal;
the inclination of each animal's leg; and/or
the leg's folding angle of each animal's leg.

### BRIEF DESCRIPTION OF THE FIGURES

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the figures:

FIG. 1 illustrates the height changes of any point on animal's body while walking.

FIG. 2 illustrates the hip's height changes of a healthy animal, in a two steps move, and the obtained repeating wave.

FIG. 3 illustrates the hip's height changes of a lameness animal, in a two steps move, and the obtained repeating wave.

### DESCRIPTION OF THE PREFERRED EMBODIMENT

The present invention is a method and a system for animal's lameness recognition.

The present invention is based on the behavior of a lameness animal in contrast with a healthy one. Lameness caused by a disease or a wound in the leg of the animal, which causes pain while the animal is step on the diseased or wounded leg. Therefore the lameness animal, while walking, instinctively shortens the stepping time on the diseased or wounded leg by accelerating the movement of the other leg.

The method and the system according to the present invention based on the recognition of the animal's walking behavior change between one step and the next step or by comparing the animal's walking behavior with a recorded walking behavior of the same animal.

The principles and operation of the method and the system for animal's lameness recognition according to the present invention may be better understood with reference to the drawing and the accompanying description.

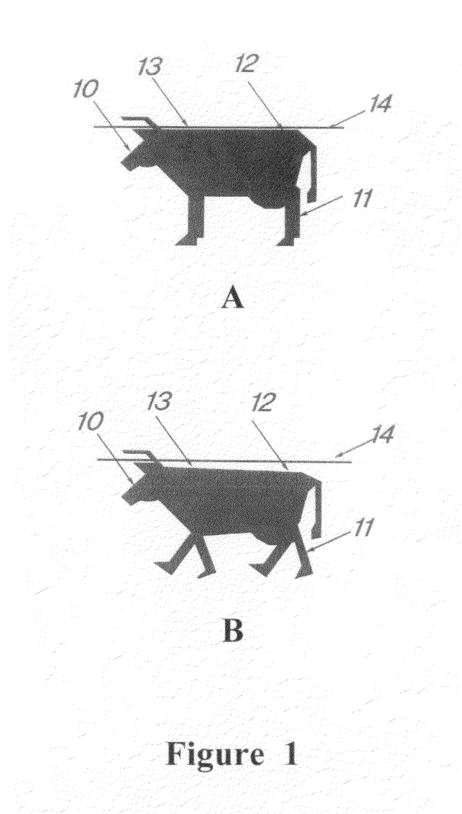

Referring now to the drawing, FIG. 1 illustrates the height changes of any point on animal's body while walking. The animal 10 is illustrated with a reference line 14. While walking, each step is done by moving one leg from behind of the other leg to the front of it. Obviously, in each step there is a situation when the legs 11 are together as illustrated in FIG. 1A. FIG. 1A illustrates the situation in which the two legs 11 are together and shows that the animal's hip 12 and the animal's withers 13 are in the highest position and almost high as the reference line 14.

FIG. 1B illustrates the animal 10 in a separated legs 11 position. This position lowers the whole body of the animal 10 lowers the hip 12 and the withers 13 as well and both are lower than the reference line 14. Therefore, from the front look on walking animal, each point on the animal's body is moving periodicity ups and down and this movement can be drawn as a repeating wave.

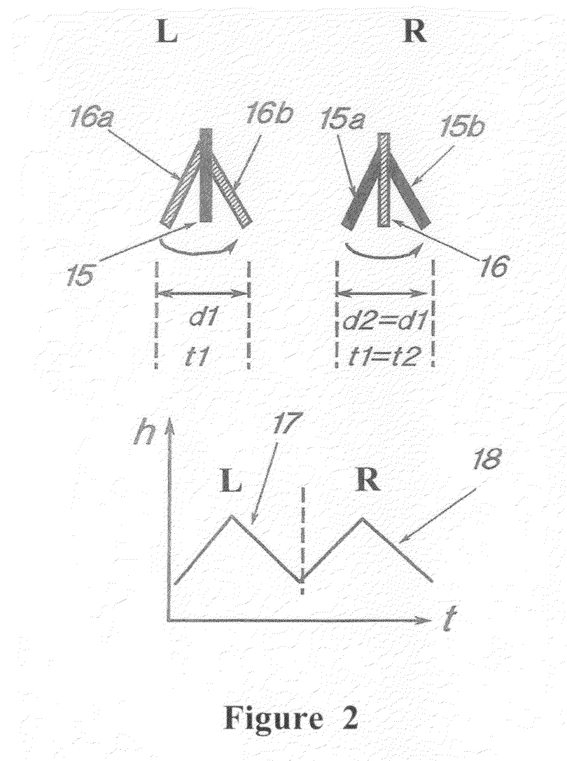

FIG. 2 illustrates the hip's height changes, during two steps move of the rear legs, of a healthy animal and the obtained repeating wave. In the left leg step L the animal moves the left leg 16 from the position 16a that is behind the right leg 15 to the position 16b that is in the front of the right leg 15. The animal doses the distance of d1 during the time of t1. The hip height changes 17 are drawn on a coordination of height (h) and time (t). In the right leg step R the animal moves the right leg 15 from the position 15a that is behind the left leg 16 to the position 15b that is in the front of the left leg 16. The animal doses the distance of d2, which usually equal or almost equal to the left leg distance d1, during the time of t2 which usually equal or almost equal to the left leg duration t1. The hip height changes 18 are drawn in the same coordination. The obtained repeating wave has the right halve 18 and the left halve 17, which looks very similar.

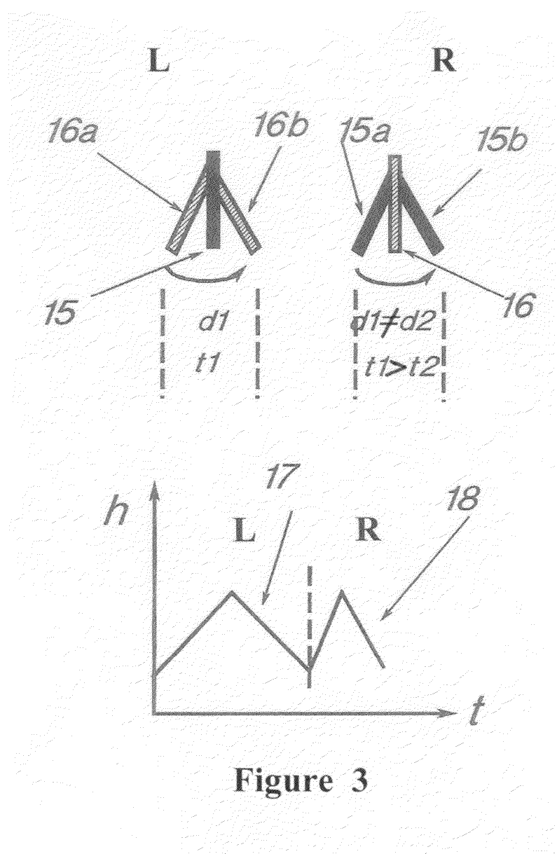

FIG. 3 illustrates the hip's height changes, during two steps move of the rear legs, of a lameness animal and the obtained repeating wave. In the left leg step L the animal moves the left leg 16a from behind the right leg 15 to the position 16b in the front of the right leg 15. The animal doses the distance of d1 during the time of t1. In the right leg step R the animal moves the right leg 15a from behind the left leg 16 to the position 15b that is in the front of the left leg 16. In this case, the animal doses the distance of d2, which usually is short than the left leg distance d1 and this difference can be also use for recognizing lameness. Moreover, because the paint of the left leg on which the animal is stepping, the animal accelerates the move of the leg and shorten the duration t2. The hip height changes 17 and 18 are drawn in the same coordination. The obtained repeating wave has the left halve 17 and the right halve 18, which are significantly changed.

The changes between the two halves of the repeating wave indicate that the animal is lameness. Same indication can be obtained from comparison of the obtained repeating wave with a pre recorded repeating wave of same animal. The lameness recognition can be done by a controller with a dedicated software or visually while the repeating wave is drawn or displayed. Moreover, an additional ID system or device enables to link between a repeating wave draw and a specific animal.

Although the invention has been described in conjunction with specific embodiments thereof, it is evident that many alternatives, modifications and variations will be apparent to those skilled in the art, accordingly, it is intended to embrace all such alternatives, modifications and variations that fall within the spirit and broad scope of the appended claims.

What is claimed is:

1. A method for recognizing a lameness of an animal, said method comprised of the following steps:
detecting position changes of a defined element on said animal's body, during a sequence of at least two said animal's steps;
comparing said position changes of an animal's step with said position changes of the subsequent step of said animal; or/and
comparing said position changes with a recorded position changes information of same element of same animal.

2. The method of claim 1, wherein lameness is defined as a significant change between said element's changes position during said animal's step and said element's change of said subsequent step and/or a significant changes between said element's changes position during said steps and said recorded element's changes.

3. The method of claim 1, further includes the step of comparing between the distance of said animal's step and the distance of the consequent step.

4. The method of claim 1, further includes the step of comparing between the back's arching of said animal with a recorded back's arching of the same animal.

5. The method of claim 1, wherein said element is the hip of said animal.

6. The method of claim 1, wherein said element is the withers of said animal.

7. The method of claim 1, wherein said element changes is at least one of the followings:
the angle between each two legs of said animal;
the hoofs distance of each two legs of said animal;
the inclination of each animal's leg; and/or
the leg's folding angle of each animal's leg.

8. The method of claim 1, wherein said detection is done by a plurality of sequential sampling of said element' position during each said animal's step to obtain a repeating wave for each two steps of said animal and wherein lameness is defined as a significant pattern's change between the two halves of said repeating wave.

9. The method of claim 1, wherein said position changes are a plurality of height or lowness measurement according to a reference.

10. The method of claim 1, wherein said detection is done by a sensor or by measuring device.

11. The method of claim 1, wherein a processor does said comparison.

12. The method of claim 1, wherein said position changes are drawn by a drawing means or displayed, and said comparison in done visually.

13. An animal' lameness recognizing system comprising:
an animal's ID means;
a detecting means for tracking position changes of an animals' body element during at lest two steps of said animal;
memory for storing said tracking information and other information; and
a controller operative for:
receiving the ID of an animal from said ID means;
receiving said element's tracking information from said tracking means or from said memory and obtaining an element's position changes repeated-wave of two following steps;
analyzing said repeated-wave to recognize lameness; and
providing announcement and animal ID, when lameness is recognized.

14. The system of claim 13, wherein said repeated-wave is analyzed by the way of:
applying mathematical technique to divide said repeated-wave into two cycle halves;
comparing the pattern of said two cycle halves; and
defining lameness in the case that said two cycle halves are significantly differ.

15. The system of claim 13, wherein said repeated-wave is analyzed by the way of:
comparing the pattern of said repeated-wave with a pre recorded repeated-wave of same animal's body element of same animal in a non-lameness condition.

16. The system of claim 13, wherein said element changes is at least one of the followings:
the angle between each two legs of said animal;
the hoofs distance of each two legs of said animal;
the inclination of each animal's leg; and/or
the leg's folding angle of each animal's leg.
